(19) United States
(12) Patent Application Publication
(10) Pub. No.: US 2025/0177289 A1
(43) Pub. Date: Jun. 5, 2025

(54) MICRO-IMPLEMENT AND METHOD OF MANUFACTURING MICRO-IMPLEMENT

(71) Applicant: Micro Complex LLC., Tokyo (JP)

(72) Inventors: Yoshikazu TOBINAGA, Otsu-shi (JP); Yasutane TAKAFUJI, Yokohama-shi (JP); Toshihiro SAITO, Tokyo (JP)

(21) Appl. No.: 18/944,770

(22) Filed: Nov. 12, 2024

(30) Foreign Application Priority Data
Nov. 30, 2023 (JP) 2023-202457

(51) Int. Cl. A61K 9/00; A61K 47/36; A61M 37/00

(52) U.S. Cl. CPC A61K 9/0021; A61K 47/36; A61M 37/0015; A61M 2037/0023; A61M 2037/0053

(57) ABSTRACT

A method for manufacturing a micro-implement according to a present embodiment is a method for manufacturing a micro-implement having one or more fine needles formed of maltose mixed with a drug, and a substrate on which the fine needles are mounted. The method includes irradiating the fine needles with plasma light to strengthen surfaces of the fine needles.

### CROSS REFERENCE TO RELATED APPLICATIONS

This application is based upon and claims the benefit of priority from the prior Japanese Patent Application No. 2023-202457 filed in Japan on Nov. 30, 2023; the entire contents of which are incorporated herein by reference.

### FIELD

An embodiment of the present invention relates to a micro-implement and a method of manufacturing the micro-implement.

### BACKGROUND

A micro-implement is a medical instrument having fine needles formed of a biological material. The fine needles are formed of maltose (malt sugar) mixed with a drug. A fine needle has a width of about 0.1 mm and a length of about 1 mm. By pressing the micro-implement against the skin, the fine needles are inserted into the skin. The fine needles formed of maltose inserted into the skin dissolve in water and penetrate into the body together with the drug mixed in the maltose. By using such a micro-implement instead of a metal or plastic fine needle, medical waste can be reduced, which is an environmental measure.

The fine needles of a micro-implement are often formed with a sharpened, cone-shaped tip to reduce pain when inserted into the skin. However, since the fine needles of the micro-implement are formed of maltose, the fine needles are prone to absorbing moisture in the air. When the maltose fine needles absorb moisture in the air, the sharp shape of the tips of some fine needles can collapse and the tips can become rounded accordingly. The tip of a fine needle thus rounded often causes increased pain to the patient when the micro-implement is pressed against the skin. In addition, it becomes impossible to administer the drug to the intended depth beneath the skin.

An object of the present invention is to prevent the tip shape of fine needles of a micro-implement from collapsing. Another object of the present invention is to shorten the time it takes for maltose, which forms the fine needles, to penetrate into the body.

### SUMMARY

In order to achieve the above objects, a method of manufacturing a micro-implement according to an embodiment is provided in which the micro-implement includes one or more fine needles formed of maltose mixed with a drug, and a substrate on which the fine needles are mounted. The method of manufacturing the micro-implement according to the embodiment includes irradiating the fine needles with plasma light to strengthen surfaces of the fine needles.

A method of manufacturing a micro-implement according to another embodiment is provided in which the micro-implement includes one or more fine needles formed of maltose mixed with a drug, and a substrate on which the fine needles are mounted. The method of manufacturing the micro-implement according to the embodiment includes irradiating the fine needles with plasma light to refine an aggregate of maltose that forms each fine needle.

### BRIEF DESCRIPTION OF THE DRAWINGS

FIG. 1 is a perspective view of a plasma generating device according to a first embodiment;

FIG. 2 is a perspective view of the device of FIG. 1 with the case removed;

FIG. 3 is a cross-sectional view of the plasma generating device according to the first embodiment;

FIG. 4 is a diagram illustrating an arrangement of second magnets according to the first embodiment;

FIG. 5 is a diagram illustrating electrical wiring of the plasma generating device according to the first embodiment;

FIG. 6 is a diagram illustrating an operation of the plasma generating device according to the first embodiment;

FIG. 7 is a conceptual diagram of a micro-implement according to the first embodiment;

FIG. 8 is a flowchart illustrating a process of improving characteristics of the fine needles of the micro-implement according to the first embodiment;

FIG. 9 is a diagram illustrating the process of improving characteristics of the fine needles of the micro-implement according to the first embodiment;

FIG. 10 is a diagram illustrating the process of improving characteristics of the fine needles of the micro-implement according to the first embodiment;

FIG. 11 is a diagram illustrating the process of improving characteristics of the fine needles of the micro-implement according to the first embodiment;

FIG. 12 is a diagram illustrating the process of improving characteristics of the fine needles of the micro-implement according to the first embodiment;

FIG. 13 is a diagram illustrating the process of improving characteristics of the fine needles of the micro-implement according to the first embodiment;

FIG. 14 is a diagram illustrating the process of improving characteristics of the fine needles of the micro-implement according to the first embodiment;

FIG. 15 is a diagram illustrating a fine needle of the micro-implement according to the first embodiment;

FIG. 16 is a diagram illustrating a fine needle of the micro-implement according to the first embodiment;

FIG. 17 is a diagram illustrating a fine needle of a micro-implement according to a second embodiment; and

FIG. 18 is a diagram illustrating a fine needle of a micro-implement according to a third embodiment.

### DETAILED DESCRIPTION

Hereinafter, embodiments will be described with reference to the drawings. In the description, an XYZ coordinate system consisting of mutually orthogonal X-axis, Y-axis, and Z-axis will be used as appropriate.

### First Embodiment

### <Plasma Generating Device>

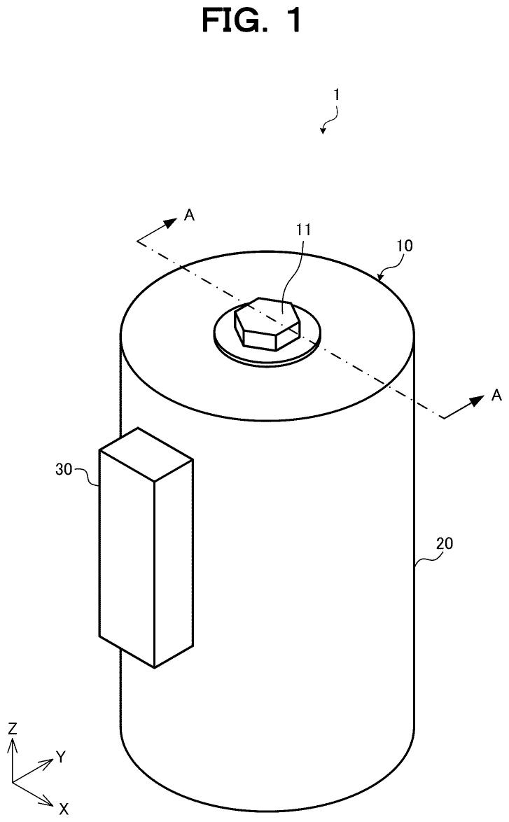

First, a plasma generating device used in a first embodiment will be described. FIG. 1 is a perspective view of the plasma generating device 1 according to the embodiment. The plasma generating device 1 includes a plasma generating unit 10 and a high-voltage power supply 30. The plasma generating unit 10 includes a cylindrical case 20 and a first electrode 11 housed in the case 20.

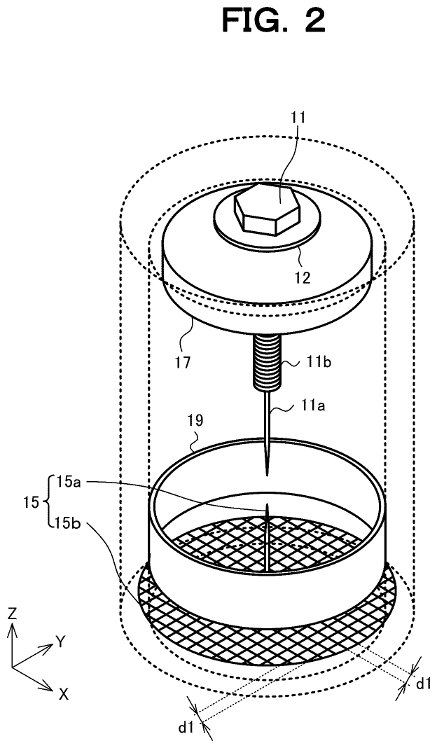

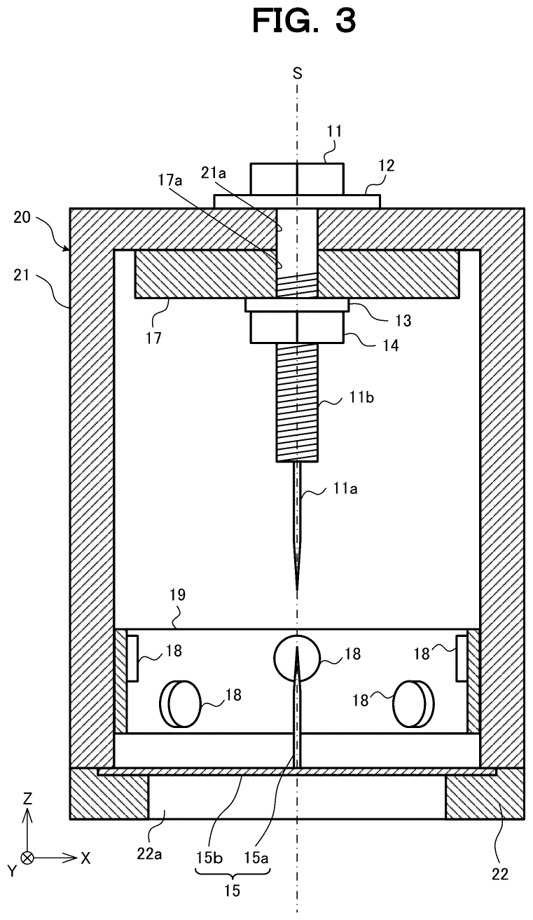

FIG. 2 is a perspective view illustrating the plasma generating unit 10 with the case 20 removed. FIG. 3 is a cross-sectional view illustrating an A-A section of FIG. 1. As illustrated in FIGS. 2 and 3, the plasma generating unit 10 has the first electrode 11, a second electrode 15, a first magnet 17, eight second magnets 18, a metal member 19, and the case 20. In FIG. 2, the second magnets 18 are not illustrated.

As illustrated in FIG. 3, the case 20 has a case body 21 and a cap 22. The case body 21 is a casing having a closed upper end and an open lower end. An opening 21a, which penetrates in the Z-axis direction, is formed in the center of an upper surface (the surface on the +Z side) of the case body 21. The case body 21 is made of, for example, resin, and has a thickness of about 4 mm, a dimension in the Z-axis direction of about 60 mm, and an inner diameter of about 40 mm. The opening 21a has an inner diameter of about 5 mm.

The cap 22 is an annular member having an opening 22a formed in its center. The cap 22 is shaped to have an outer diameter equal to the outer diameter of the case body 21, and is fixed to the end of the case body 21 on the −Z side. The cap 22 is made of, for example, resin.

As illustrated in FIG. 3, the first electrode 11 is a member whose longitudinal direction is defined as the Z-axis direction and which has two parts: a first discharge part 11a and a conductive part 11b. The conductive part 11b is an M5 size bolt having a male thread formed at its lower end. The first discharge part 11a is a member having a diameter of 1 mm and a length of about 20 mm and having a sharp lower end. The first discharge part 11a is integrated with the conductive part 11b by having its upper end welded to the lower end of the conductive part 11b. The first discharge part 11a and the conductive part 11b, which constitute the first electrode 11, are made of a material such as iron or stainless steel.

The first magnet 17 is a circular plate-shaped member. A through hole 17a, which penetrates in the Z-axis direction, is formed in the center of the first magnet 17. The first magnet 17 is, for example, a magnet with strong magnetic force, such as a neodymium magnet. The first magnet 17 has a thickness of about 5 mm and an outer diameter of about 30 mm. The through hole 17a has an inner diameter of about 5 mm. The first magnet 17 is magnetized so that its upper surface (the surface on the +Z side) is an S pole and its lower surface (the surface on the −Z side) is an N pole.

As illustrated in FIG. 3, the first electrode 11 configured as described above is inserted into the opening 21a from above the case body 21 through a washer 12. With the first electrode 11 protruding into the inside of the case body 21 inserted into the through hole 17a of the first magnet 17, the case body 21, the first electrode 11, and the first magnet 17 are integrated by engaging a washer 13 and a nut 14 with the conductive part 11b.

As illustrated in FIGS. 2 and 3, the second electrode 15 has a second discharge part 15a and a conductive part 15b. The conductive part 15b is a mesh made of metal. The conductive part 15b can be formed, for example, by cutting a metal mesh into a circular shape. The diameter of the conductive part 15b is slightly larger than the inner diameter of the case body 21, and is about 45 mm. The conductive part 15b has metal wires with an arrangement pitch d1 of about 5 mm. The metal wire has an outer diameter of about 0.2 mm. The second discharge part 15a is welded to the center of the conductive part 15b.

The second discharge part 15a is a member having a diameter of 1 mm and a length of about 8 mm and having a sharp upper end. The second discharge part 15a is integrated with the conductive part 15b by having its lower end welded to the center of the conductive part 15b. The second discharge part 15a and the conductive part 15b, which constitute the second electrode 15, are made of a material such as iron or stainless steel.

The second electrode 15 configured as described above is assembled to the case 20 by sandwiching the outer edge of the conductive part 15b between the case body 21 and the cap 22. In this state, the first discharge part 11a of the first electrode 11 and the second discharge part 15a of the second electrode 15 are arranged on a straight line S parallel to the Z axis, as illustrated in FIG. 3. The tip of the first discharge part 11a and the tip of the second discharge part 15a face each other with a predetermined gap therebetween, thereby forming a discharge gap.

The metal member 19 is a cylindrical member. The metal member 19 is formed by bending a metal plate with a thickness of 1 mm to fit the inner peripheral surface of the case body 21. The metal member 19 has a height (the dimension in the Z-axis direction) of about 10 mm and an outer diameter approximately equal to the inner diameter of the case body 21. The metal member 19 is attached to the case body 21 by, for example, bonding the outer peripheral surface to the inner peripheral surface of the case body 21. The metal member 19 is made of a metal that is attracted to a magnet, such as iron.

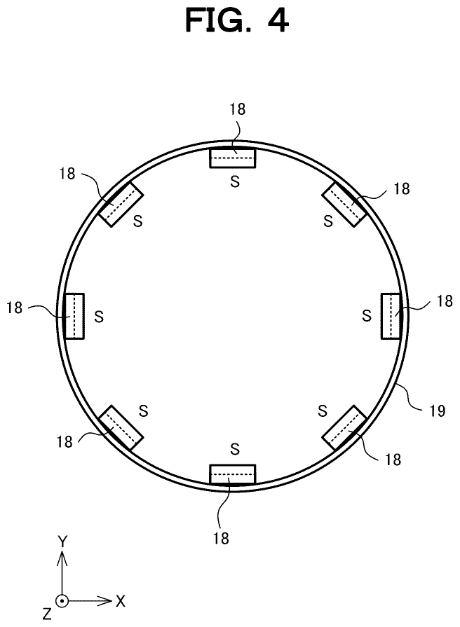

Each of the eight second magnets 18 is a magnet with strong magnetic force, such as a neodymium magnet. Each of the second magnets 18 is shaped like a circular plate having a diameter of about 4 mm. Each of the second magnets 18 is magnetized so that one surface thereof is an S pole and the other surface thereof is an N pole. Each of the second magnets 18 is attached to the metal member 19 by bonding the surface on which the N pole appears to the metal member 19. The second magnets 18 are arranged at equal intervals along the inner peripheral surface of the metal member 19 with the straight line S as the center. The second magnets 18 are also arranged so that adjacent second magnets are vertically offset from each other. As illustrated in FIG. 4, the second magnets 18 are arranged so that the surfaces on which the S pole appears face each other.

In the plasma generating device 1, as illustrated in FIGS. 2 and 3, the metal member 19 is disposed below the discharge gap between the tip of the first discharge part 11a and the tip of the second discharge part 15a. Accordingly, the plurality of second magnets 18 are arranged so as to surround the second discharge part 15a.

As illustrated in FIG. 1, the high-voltage power supply 30 is fixed to the case body 21. The high-voltage power supply 30 is, for example, a power supply including a DC/DC converter. The high-voltage power supply 30 is connected to a DC power supply 100 that converts power from a commercial power supply into DC power, for example. The high-voltage power supply 30 boosts the output voltage output from the DC power supply 100 and outputs the resulting voltage to the plasma generating unit 10. The DC power supply 100 to be used may be one for an output voltage of about 3 V to 5 V.

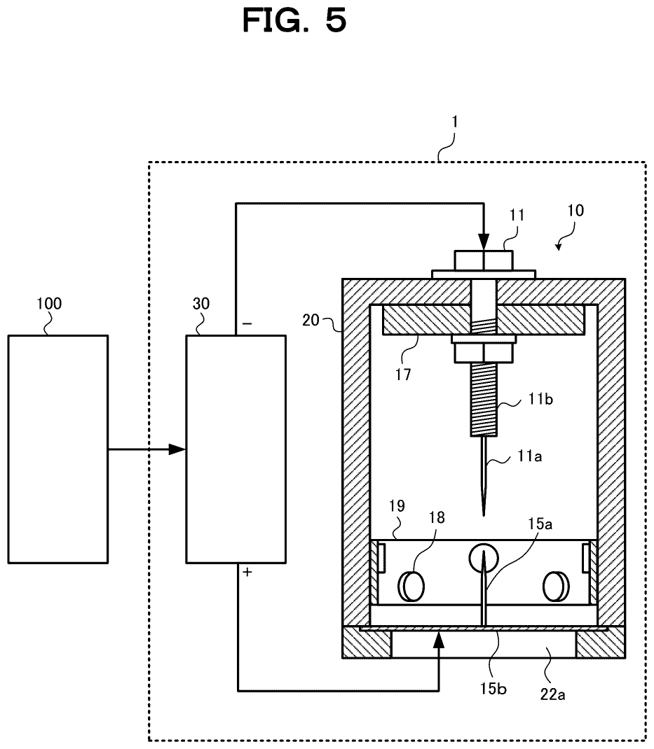

FIG. 5 is a diagram illustrating electrical wiring of the plasma generating device 1. As illustrated in FIG. 5, the high-voltage power supply 30 has a negative electrode connected to the first electrode 11 and a positive electrode connected to the second electrode 15. The high-voltage power supply 30 applies a DC voltage of 50,000 V to 1,000,000 V to the first electrode 11 and the second electrode 15. This causes an arc discharge to occur in the discharge gap between the first discharge part 11a and the second discharge part 15a. The output voltage of the high-voltage power supply 30 is adjusted according to conditions such as the distance between the first discharge part 11a and the second discharge part 15a, the shapes of the tips of the first discharge part 11a and the second discharge part 15a, the air pressure, and humidity. The output voltage of the high-voltage power supply 30 is adjusted herein, for example, to about 400,000 V.

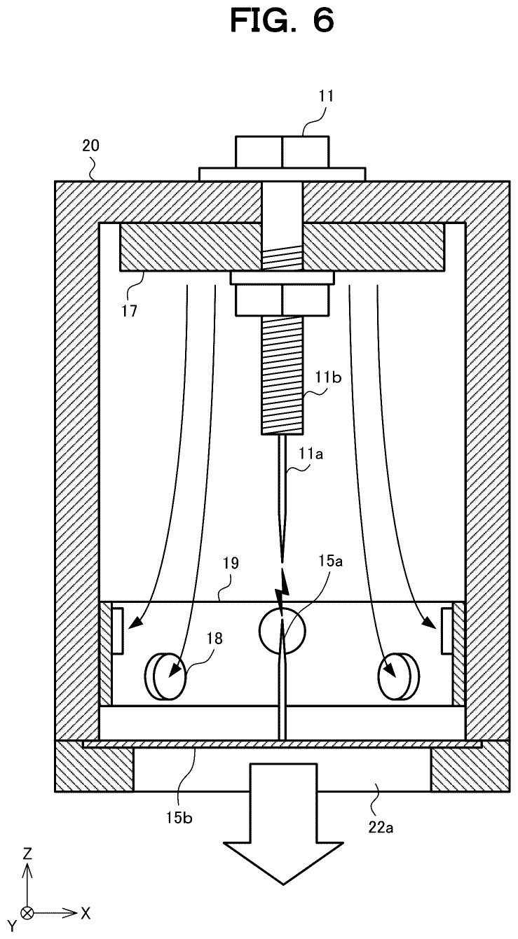

Next, an operation of the plasma generating device 1 will be described with reference to FIG. 6. The metal member 19 is magnetized by the second magnet 18. Specifically, since the N pole of the second magnet 18 is in magnetic contact with the metal member 19, the inner peripheral surface of the metal member 19 that is in contact with the N pole of the second magnet 18 is magnetized to an S pole.

As illustrated by arrows in FIG. 6, inside the case body 21, a magnetic field is formed that flows from the N pole of the first magnet 17 toward the S pole of the second magnet 18 and the inner peripheral surface of the metal member 19. Since the second magnet 18 and the metal member 19 are located closer to the −Z side than the tip of the first discharge part 11a on the −Z side is, a magnetic field in a direction toward the opening 22a (−Z direction) is formed in the vicinity of the first discharge part 11a and the second discharge part 15a. At and in the vicinity of the opening 22a, a portion of the magnetic field is formed toward the second magnet 18 and the metal member 19, while most of the magnetic field is formed from the second discharge part 15a toward the opening 22a.

When the DC voltage is output from the DC power supply 100, the high-voltage power supply 30 outputs a DC voltage of about 400,000 V. As a result, a DC voltage of about 400,000 V is applied between the first electrode 11 and the second electrode 15. Accordingly, an arc is generated between the first discharge part 11a and the second discharge part 15a. When an arc is generated, some molecules that make up the atmosphere around the first discharge part 11a and the second discharge part 15a are separated into positive ions and electrons, generating plasma. The amount of plasma generated can be adjusted by the output power of the high-voltage power supply 30, the voltage applied between the first electrode 11 and the second electrode 15, the distance between the first electrode 11 and the second electrode 15, and the like.

As described above, inside the case body 21, a magnetic field is generated in a direction from the first discharge part 11a and the second discharge part 15a toward the opening 22a. Plasma has a property of moving along magnetic field lines that indicate the direction of a magnetic field. Therefore, most of the generated plasma does not remain inside the case 20, but is emitted to the outside of the case 20 through the opening 22a. A micro-implement 200, which is a target to be irradiated with plasma, is disposed near the opening 22a of the plasma generating device 1.

### <Micro-Implement>

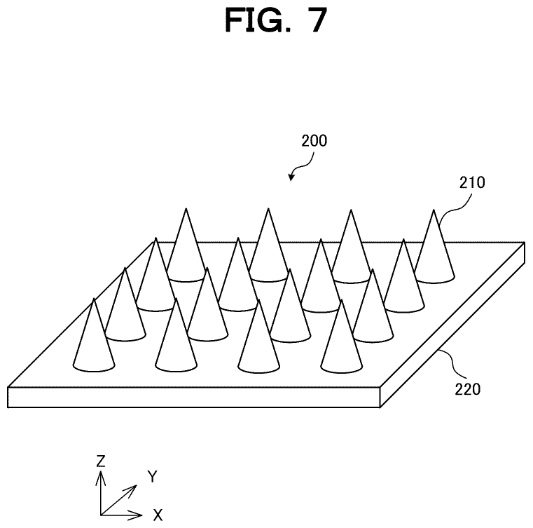

Next, a configuration of the micro-implementation will be described. FIG. 7 is a perspective view illustrating an example of the micro-implement 200. The micro-implement 200 has one or more fine needles 210 formed of maltose mixed with a drug, and a substrate 220 on which the fine needles 210 are mounted. The substrate 220 has, for example, a length of about 10 mm in the X-axis and Y-axis directions and a length of about 1 mm in the Z-axis direction. Each fine needle 210 has a diameter of about 0.1 mm and a length of about 1 mm. In FIG. 7, 16 fine needles 210 are illustrated, but the number of fine needles 210 is not limited. The substrate 220 is formed of, for example, plastics such as polyvinyl alcohol, pullulan, and polyethylene glycol, and biological materials including, for example, polysaccharides such as hyaluronic acid or proteins such as collagen.

### <Method of Manufacturing Micro-Implement>

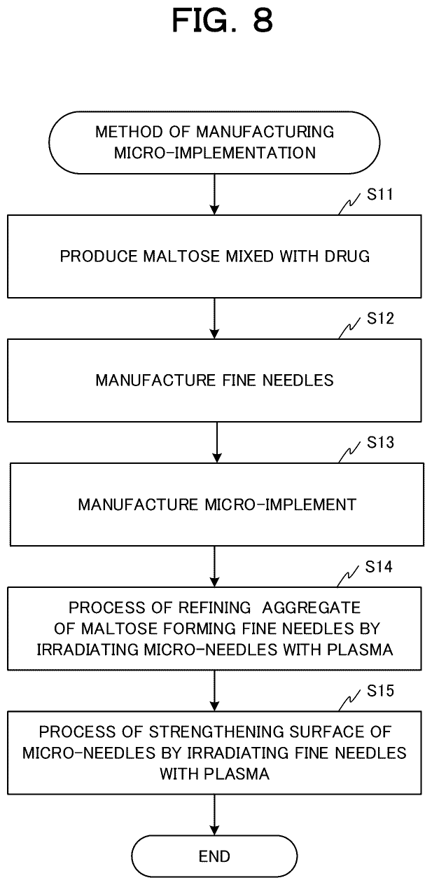

Next, a method of manufacturing the micro-implement 200 will be described with reference to FIG. 8.

First, maltose mixed with a drug is produced (step S11). Specifically, the drug is mixed into commercially available powdered maltose. The drug (pharmaceutical agent or drug used in cosmetics) to be mixed with maltose may be water-soluble. Preferred pharmaceutical agents include many agents, including local anesthetics such as lidocaine. Particularly effective are polymeric pharmaceutical agents. Examples of the polymeric pharmaceutical agents include physiologically active peptides and their derivatives, nucleic acids, oligonucleotides, various antigen proteins, bacteria, and virus fragments. Examples of the physiologically active peptides and their derivatives include calcitonin, adrenocorticotropic hormone, parathyroid hormone (PTH), hPTH (1->34), EGF, insulin, secretin, luteinizing hormone-releasing hormone, growth hormone, growth hormone-releasing hormone, thyroid-stimulating hormone, prolactin, interferon, interleukin, G-CSF, endothelin, and salts thereof. Examples of the antigen proteins include HBs surface antigen and HBe antigen. Examples of the above-mentioned drugs used in cosmetics include whitening ingredients such as kojic acid, rucinol, tranexamic acid, and vitamin A derivatives; anti-wrinkle ingredients such as retinol, retinoic acid, retinol acetate, retinol palmitate; blood circulation promoting ingredients such as capsine and vanillylamide norylate; diet ingredients such as raspberry ketone, evening primrose extract, and seaweed extract; antibacterial ingredients such as isopropyl methylphenol, photosensitizer, and zinc oxide; and vitamins such as vitamin D2, vitamin D3, and vitamin K.

Next, fine needles 210 having a predetermined size and shape are manufactured using the maltose produced in step S11 (step S12). Specifically, powdered maltose mixed with the drug is placed into a mold for the fine needles 210. Then, the powdered maltose is heated to melt it. Maltose in a molten state is sometimes called liquid phase maltose. Next, the liquid phase maltose contained in the mold for the fine needles 210 is cooled and solidified. Maltose in a solid state is sometimes called coagulated (solid-phase) maltose.

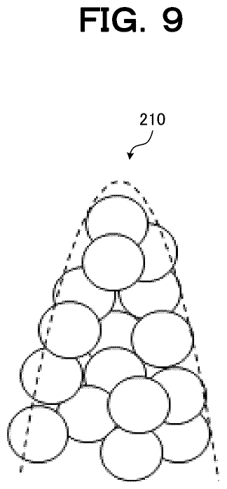

FIG. 9 is a conceptual diagram of an aggregate of maltose (particles) forming a fine needle 210. The size of the aggregate of maltose that forms a fine needle 210 manufactured in the process of step S12 is about 0.1 μm to 1.0 μm. The maltose material that has been melted and then cooled contains aggregates of a plurality of molecules. The size of the aggregate of maltose that forms the fine needle 210 after the process in step S12 is considerably larger than the size of a single maltose molecule.

Next, a plurality of fine needles 210 are mounted on the substrate 220 to manufacture the micro-implement 200 as illustrated in FIG. 7 (step S13).

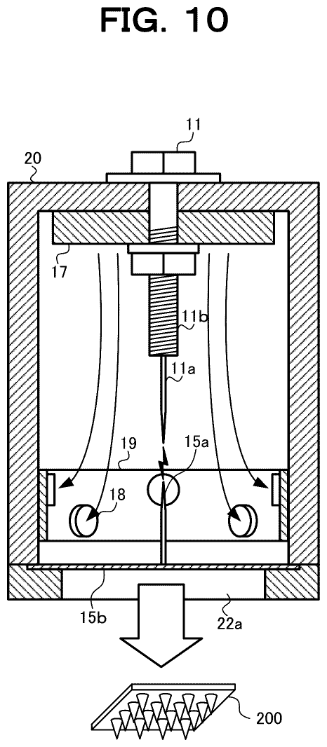

Next, a process of refining an aggregate contained in the maltose material is performed (step S14). As illustrated in FIG. 10, the micro-implement 200 is placed near the opening 22a of the plasma generating device 1, and plasma light is emitted from, for example, the substrate 220 (rear surface) side of the micro-implement 200.

Plasma has a property of emitting light with wavelengths as short as X-rays by an arc discharge that generates energy of several tens of KeV. Therefore, rather than saying “irradiated with plasma light”, it may be more accurate to say “irradiated with light of wavelengths as short as X-rays accompanying the generation of plasma”. Plasma has a collective phenomenon in which electrons liberated from ion molecules vibrate with each other, and also has the wave properties of high-frequency light. Accordingly, the way to express plasma depends on which characteristic is being focused on. Expressions such as “irradiated with plasma” and “irradiated with plasma light” will be used herein.

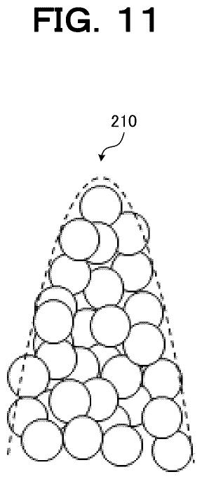

By irradiating maltose that forms a fine needle 210 with short-wavelength (high-frequency) plasma light having high energy, maltose molecules that form the fine needle 210 are excited. The excited maltose molecules have weaker cohesive forces with adjacent molecules, making it difficult for many molecules to aggregate together to form a large aggregate. Therefore, by being irradiated with the plasma light, the aggregate of maltose that forms the fine needle 210 can be refined. FIG. 11 is a conceptual diagram illustrating that an aggregate of maltose that forms the fine needle 210 has been refined by being irradiated with plasma light. By being irradiated with plasma light, the aggregate of maltose illustrated in FIG. 11 has a smaller size than the size of the aggregate before irradiated with plasma light illustrated in FIG. 9. The size of the aggregate of maltose that forms the fine needle 210 manufactured in the process of step S14 can be suppressed to 0.01 μm to 0.1 μm.

The rate at which the particles are refined varies depending on the amount of plasma (strength of the plasma light) emitted from the plasma generating device used, but the aggregate of maltose can be refined by being irradiated with plasma light. The irradiation time of the plasma light is determined based on the relationship between the required degree of particle refining, the intensity of plasma light for irradiation, the required rate at which the fine needle 210 dissolves in the body (size of the particles that form maltose), and the irradiation time of the plasma light (manufacturing cost), among other factors. The irradiation time of the plasma light in step S14 is, for example, about 15 to 30 minutes.

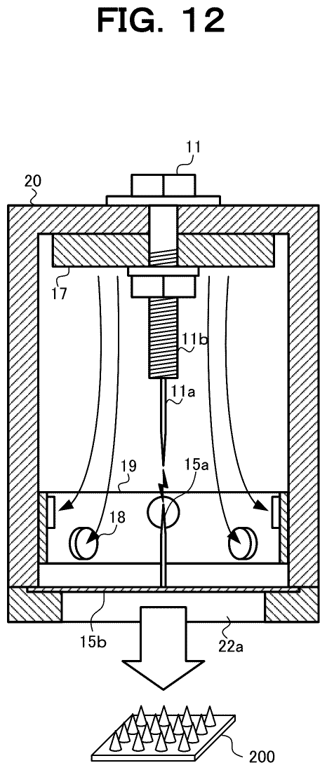

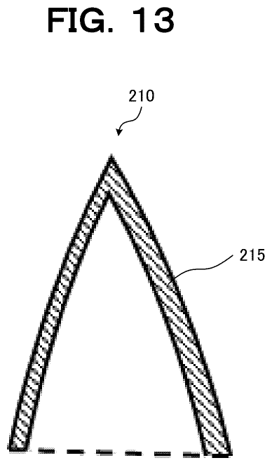

Next, a process of strengthening (hardening) the surface of the fine needle 210 is performed by irradiating the fine needle 210 with plasma light (step S15). For example, as illustrated in FIG. 12, the micro-implement 200 is placed near the opening 22a of the plasma generating device 1, and plasma light is emitted from the fine needle 210 (surface) side of the micro-implement 200. By being irradiated with the plasma light, a polymerized coating layer 215 is formed on the surface of the fine needle 210 as illustrated in FIG. 13.

The aggregate of maltose on the surface of the fine needle 210 irradiated with plasma light undergoes a reaction between oxygen in the atmosphere and hydrogen that constitutes the maltose, removing moisture and resulting in a state of high carbon concentration. In this state, the tendency for carbon molecules to bond together increases, resulting in polymerization (bonding) of maltose molecules. As a result, a polymerized coating layer 215 formed of polymerized maltose molecules, including sugarcoating in humid atmosphere, is formed on the surface of the fine needle 210.

The rate at which the surface of maltose is strengthened varies depending on the amount of plasma (strength of the plasma light) emitted from the plasma generating device used, but the surface of maltose can be strengthened by being irradiated with plasma light. The required strength of maltose also varies depending on the storage conditions (temperature, humidity, etc.) of the micro-implement 200. The irradiation time of plasma in step S15 is, for example, about 5 to 15 minutes.

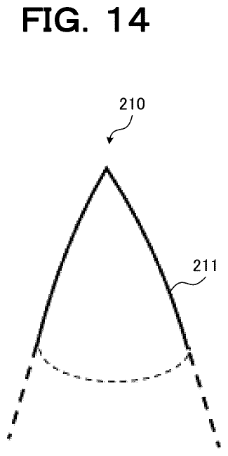

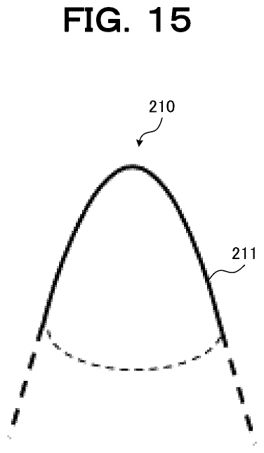

FIG. 14 is a conceptual diagram illustrating a tip part 211 of the fine needle 210 of the micro-implement 200 manufactured through the process up to step S14. Maltose easily absorbs moisture in the air. When maltose absorbs moisture in the air, the sharp shape of the tip of the fine needle 210 can collapse and the tip can become rounded accordingly as illustrated in FIG. 15. If the tip of the fine needle 210 has a rounded shape, it often causes more pain to the patient when the micro-implement 200 is pressed against the skin.

By performing the process of step S15, the polymerized coating layer 215 is formed on the surface of the fine needle 210 as illustrated in FIG. 13. This is due to the ultraviolet strengthening properties of a certain resin. The formation of the polymerized coating layer 215, with sugar coating, can prevent the maltose that forms the fine needle 210 from absorbing moisture in the surroundings. This makes it possible to prevent the tip shape of the fine needle 210 of the micro-implement 200 from collapsing.

As described above, in the micro-implement 200 according to the embodiment, by irradiating the fine needle 210 with plasma light, the aggregate of maltose that forms the surface of the fine needle 210 form the polymerized coating layer 215, with sugar coating, thereby strengthening the surface of the fine needle 210. This makes it possible to prevent the tip shape of the fine needle 210 of the micro-implement 200 from collapsing.

In addition, in the micro-implement 200 according to the embodiment, by irradiating the fine needle 210 with plasma light, the aggregate of maltose that forms the fine needle 210 is excited by the high energy of the short-wavelength plasma light, thereby causing the aggregate of maltose to be refined. The smaller the aggregate of maltose, the more easily the maltose can be penetrated into the body. Therefore, by refining the aggregate of maltose, the time required for the drug contained in maltose to be penetrated into the human body can be shortened. In addition, in a case where drugs are mixed in, if organic molecules such as amino groups or amine molecules, or biological materials including, for example, polysaccharides like hyaluronic acid and proteins like collagen are mixed in, they are organic molecules containing nitrogen atoms and thus are softened by ultraviolet light, and the aggregate containing these molecules becomes even finer, improving their penetrativeness into the body and enhancing their medicinal efficacy.

By being irradiated with plasma light, the aggregate of maltose inside the fine needle 210 is refined. On the other hand, the maltose on the surface of the fine needle 210 forms the polymerized coating layer 215. In this way, by being irradiated with plasma light, the aggregate of maltose can be refined and the surface of the fine needle 210 can be strengthened.

In the micro-implement 200 according to the embodiment, since the fine needle 210 is formed of maltose, the fine needle 210, even when breaking inside the body, will not cause a medical accident. In addition, since the micro-implement 200 has the fine needle(s) 210 formed of maltose, it is possible to reduce medical waste, which contributes to environmental conservation.

A case has been described above in which the micro-implement 200 is irradiated with plasma light using a small plasma generating device 1 as illustrated in FIG. 1 and others. However, the plasma generating device to be used does not need to be limited to the plasma generating device 1 as illustrated in FIG. 1 and others. For example, a large plasma generating device that is AC powered may be used.

In addition, a case has been described above in which, in the process of refining maltose particles in step S14, plasma light is emitted from the substrate 220 side of the micro-implement 200, and in the process of strengthening the surface of the fine needle(s) 210 in step S15, plasma light is emitted from the fine needle 210 side of the micro-implement 200. However, the process of refining the aggregate of maltose and the process of strengthening the surface of the fine needle(s) 210 can be performed in one process. For example, these processes may be replaced with a process of emitting plasma light from the fine needle 210 side of the micro-implement 200. For example, as illustrated in FIG. 12, the surface (fine needle 210 side) of the micro-implement 200 is irradiated with plasma light. In this case, the plasma light is emitted until the polymerized coating layer 215 is formed on the surface of the fine needle(s) 210.

A case has been described above in which the shape of each fine needle 210 is a cone. However, the shape of the fine needle 210 does not need to be limited to a cone. For example, the shape of the fine needle 210 may be a pyramidal shape such as a triangular pyramid or a quadrangular pyramid.

Further, a case has been described above in which each fine needle 210 has a width of about 0.1 mm and a length of about 1 mm. However, the width and length of the fine needle 210 do not need to be limited to these values. The width and length of the fine needle 210 can be adjusted taking into consideration the depth from the skin surface to which the drug is to be delivered, the amount of drug to be delivered, and the degree of pain felt when the micro-implement 200 is pressed against the skin. For example, when the micro-implement 200 is used for a horse or the like, a thick and long fine needle(s) 210 may be used.

### Second Embodiment

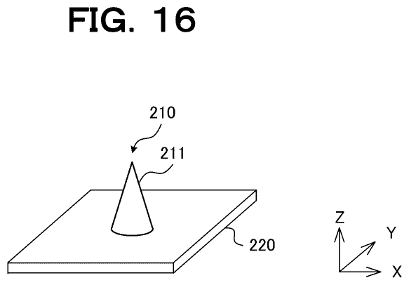

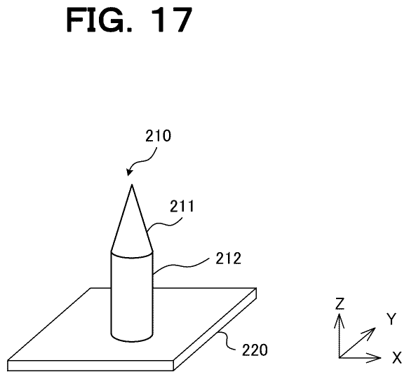

FIG. 16 is a diagram focusing on one of the fine needles 210 illustrated in FIG. 7. In the first embodiment, each fine needle 210 has been described as having a conical shape. A fine needle 210 according to a second embodiment is illustrated in FIG. 17. The fine needle 210 of the micro-implement 200 according to the second embodiment has a tip part 211 and a cylindrical support part 212 that is provided between the tip part 211 and a substrate 220 and has the same diameter as the tip part 211. By changing the mold used in the process of step S12 illustrated in FIG. 8, the fine needle 210 having the shape illustrated in FIG. 17 can be manufactured. By providing the support part 212, the length from the substrate 220 to the tip of the fine needle 210 can be adjusted. This allows the depth from the skin surface to which the drug is delivered by the fine needle 210 to be adjusted.

### Third Embodiment

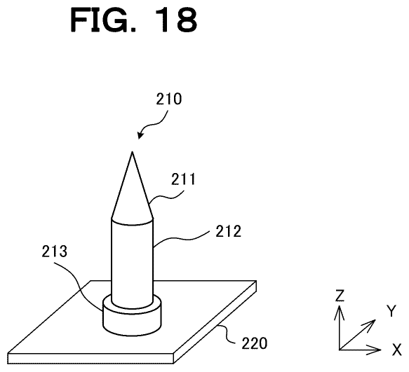

A fine needle 210 of a micro-implement 200 according to a third embodiment is illustrated in FIG. 18. The fine needle 210 according to the third embodiment has a conical tip part 211, a support part 212 as described in the second embodiment, and a cylindrical base part 213 that is provided between the support part 212 and a substrate 220 and has a diameter larger than the diameter of the support part 212. By changing the mold used in the process of step S12 illustrated in FIG. 8, the fine needle 210 having the shape illustrated in FIG. 18 can be manufactured. By providing the base part 213, the depth from the skin surface to which the drug is delivered by the fine needle 210 can be adjusted.

Although several embodiments of the present invention have been described, these embodiments are presented as examples and are not intended to limit the scope of the invention. These novel embodiments can be embodied in various other forms, and various omissions, substitutions, and modifications can be made without departing from the scope and spirit of the invention. These embodiments and their variations are included in the scope and spirit of the invention, and are included in the scope of the invention and its equivalents described in the claims.

1. A method for manufacturing a micro-implement having one or more fine needles formed of maltose mixed with a drug, and a substrate on which the fine needles are mounted, the method including the step of:
irradiating the fine needles with plasma light to strengthen surfaces of the fine needles.

2. A method for manufacturing a micro-implement having one or more fine needles formed of maltose mixed with a drug, and a substrate on which the fine needles are mounted, the method including the step of:
irradiating the fine needles with plasma light to refine an aggregate of maltose that forms each fine needle.

3. A method for manufacturing a micro-implement having one or more fine needles formed of maltose mixed with a drug, and a substrate on which the fine needles are mounted, the method including the step of:
irradiating the fine needles with plasma light to strengthen surfaces of the fine needles by using a plasma generating device that irradiates a target with the plasma light,
wherein the plasma generating device includes a first electrode to which a negative electrode of a direct current power supply is applied, and a second electrode to which a positive electrode of the direct current power supply is applied, and the first electrode and the second electrode are arranged so that each tip thereof face each other, and
wherein the plasma generating device includes a first magnet and a second magnet that are positioned mutually separated from each other and form a magnetic field in a direction toward the target around the tip of the first electrode and the tip of the second electrode, which are mutually opposed.

4. A method for manufacturing a micro-implement having one or more fine needles formed of maltose mixed with a drug, and a substrate on which the fine needles are mounted, the method including the step of:
irradiating the fine needles with plasma light to refine an aggregate of maltose that forms each of the fine needle by using a plasma generating device that irradiates a target with the plasma light,
wherein the plasma generating device includes a first electrode to which a negative electrode of a direct current power supply is applied, and a second electrode to which a positive electrode of the direct current power supply is applied, and the first electrode and the second electrode are arranged so that each tip thereof face each other, and
wherein a first magnet and a second magnet that are positioned mutually separated from each other and form a magnetic field in a direction toward the target around the tip of the first electrode and the tip of the second electrode, which are mutually opposed.

5. A micro-implement having one or more fine needles formed of maltose mixed with a drug, and a substrate on which the fine needles are mounted,
wherein a polymerized film layer is formed on the surface of the fine needles in which maltose aggregates are polymerized by irradiating the fine needles with plasma light.

6. A micro-implement having one or more fine needles formed of maltose mixed with a drug, and a substrate on which the fine needles are mounted,
wherein an aggregate of the maltose that forms each of the fine needles, is refined by irradiating the fine needles with plasma light.

7. The micro-implement according to claim 6, wherein the size of the maltose aggregates refined by the plasma light irradiation is in the range of 0.01 μm to 0.1 μm.

8. The micro-implement according to claim 5, wherein each of the fine needles comprises a conical tip part, and a cylindrical support part having the same diameter thereof as that of the tip part provided between the tip part and a substrate on which the fine needles are mounted.

9. The micro-implement according to claim 8, wherein each of the fine needles further comprises a cylindrical base part that is provided between the support part and the substrate and a diameter thereof is larger than that of the support part.
